(19) United States
(12) Patent Application Publication
(10) Pub. No.: US 2010/0121210 A1
(43) Pub. Date: May 13, 2010

(54) METHOD AND DEVICE FOR DETECTION OFANALYTE IN VAPOR OR GASEOUS SAMPLE

(75) Inventors: Erno Lindner, Germantown, TN (US); Jidong Guo, Changchun City (CN)

Correspondence Address: NIXON PEABODY LLP - PATENT GROUP, 1100 CLINTON SQUARE, ROCHESTER, NY, 14604, US

(73) Assignee: UNIVERSITY OF MEMPHIS RESEARCH FOUNDATION, Memphis, TN (US)

(21) Appl. No.: 12/579,863

(22) Filed: Oct. 15, 2009

Related U.S. Application Data
(60) Provisional application No. 61/105,788, filed on Oct. 15, 2008.

(51) Int. Cl. A61B 5/08; C12Q 1/24; C25B 11/00; G01N 27/26

(52) U.S. Cl. 600/532; 204/403.14; 205/775; 205/785.5

(57) ABSTRACT

A method and system for electrochemical detection of an analyte in a vapor or gas sample is provided. The method and system include exposing a vapor or gas sample to an electrochemical sensor comprising one or more electrodes and a coating that surrounds the one or more electrodes, which coating is capable of partitioning the analyte directly from the vapor or gas sample. The method and system include detecting an oxidation/reduction current during the exposing, wherein the detected current relates to a concentration of analyte in the vapor or gas sample.

### CROSS REFERENCE TO RELATED APPLICATIONS

This application claims the benefit of U.S. Provisional Patent Application Ser. No. 61/105,788, filed on Oct. 15, 2008, which is hereby incorporated by reference in its entirety.

### FIELD OF THE INVENTION

Various aspects of the invention generally relate to a method and system for detection of an analyte, for example, a hydrophilic analyte, in a vapor or gaseous sample, and more specifically to a corresponding method and system for detecting Streptococcus pneumoniae infection.

### BACKGROUND OF THE INVENTION

Electrochemistry is a branch of chemistry that deals with the current produced by an electron transfer reaction of an electrochemically active species at the surface of a conductive electrode driven by an externally applied potential. Electrochemistry is a powerful tool in analytical chemistry to detect various compounds, for example, hydrogen peroxide, but traditionally this tool has been limited to detection of compounds present in an aqueous solution.

It has been known that Streptococcus pneumoniea bacterium present in the upper respiratory system of human beings and other mammals produce hydrogen peroxide. The amount of hydrogen peroxide produced by Streptococcus pneumoniea is a direct indicator of the population of Streptococcus pneumoniea present in a human body. Electrochemical analysis can give detailed information about the concentration of hydrogen peroxide in solution, but traditionally has not been used to study gaseous or vapor phase hydrogen peroxide present, for example, in an exhaled breath of a patient. Although Streptococcus pneumoniea is a common, innocuous inhabitant of the upper respiratory system of healthy humans, excessive undesirable amounts of this bacterium is the leading cause of infectious disease in young children and the elderly as documented, for example, in Tomasz, “Streptococcus pneumoniae: Molecular Biology and Mechanisms of Disease,” ISBN 0-913113-85-9, Mary Ann Liebert, Inc., Larchmont, N.Y. (2000). A healthy immune system keeps the bacteria population safely under control, but if the immune system is weakened, the bacteria can become invasive and cause diseases like pneumonia, otitis media, and meningitis.

Conventional treatments for Streptococcus pneumoniae infection are very harsh and invasive where a person infected with an over-population of this bacterium has his or her immune system effectively shutdown. Risk of infection in patients with this weakened immune state is high, and without the body's natural defenses to check the invading Streptococcus pneumoniae, even a mild infection can become dangerous to the point of fatality as documented in Tuomanen, “The Biology of Pneumococcal Infection,” Pediatric Research 42:253-258 (1997). Further, because Streptococcus pneumoniae is able to adapt and mutate to become resistant to antibiotics, it is not desirable to administer this treatment unless infection becomes a problem as documented in Schmidt, “Genes and Antibiotic Resistance,” Genome New Network (2000).

Conventional systems and methods for monitoring and diagnosis of Streptococcus pneumoniae levels require clinical symptoms to first become apparent, and then the diagnosis involves the use of invasive techniques to detect presence of Streptococcus pneumoniae.

Unfortunately, there is no direct method or system to detect amount of hydrogen peroxide produced by the Streptococcus pneumoniae.

What is needed, therefore, is a non-invasive technique that can rapidly detect an analyte, for example, hydrogen peroxide produced by Streptococcus pneumoniae, in a vapor or gas phase sample, such as a patient's breath.

The present invention is directed to overcoming these and other deficiencies in the art.

### SUMMARY OF THE INVENTION

A first aspect of the present invention relates to a method for electrochemical detection of an analyte in a vapor or gas sample, the method including exposing a vapor or gas sample to an electrochemical sensor comprising one or more electrodes and a coating that surrounds the one or more electrodes, which coating is capable of partitioning the analyte directly from the vapor or gas sample. The method includes detecting an oxidation/reduction current during the exposing, wherein the detected current relates to a concentration of analyte in the vapor or gas sample.

A second aspect of the present invention relates to a method of detecting a Streptococcus pneumoniae infection in a patient, the method including exposing a patient breath sample to an electrochemical sensor including one or more electrodes and a coating that surrounds the one or more electrodes, which coating is capable of partitioning hydrogen peroxide directly from the breath sample; and detecting an oxidation/reduction current during said exposing, wherein the detected current relates to a concentration of hydrogen peroxide in the patient breath sample, the concentration of hydrogen peroxide indicating the extent of the Streptococcus pneumoniae infection.

A third aspect of the present invention relates to an electrochemical sensor that includes two or more electrodes, and a coating that surrounds the two or more electrodes and is capable of selectively partitioning an analyte, e.g., hydrogen peroxide, from a vapor or gas phase such that an oxidation/reduction current within the coating can be measured.

The accompanying examples set forth herein demonstrate the development and testing of a hydrogen peroxide electrochemical sensor and detector device using a coated electrochemical sensor whose coating is capable of selectively partitioning hydrogen peroxide from a vapor or gas phase (e.g., patient breath sample). Because the partitioned analyte concentration in the coating is in equilibrium with the vapor or gas phase, i.e., the analyte concentration in the coating is proportional to the concentration in the vapor or gas phase, a reliable assessment can be made concerning the analyte concentration in the vapor or gas phase. In addition, by correlating the vapor or gas phase analyte concentration to the severity of Streptococcus pneumoniae, it is possible to assess the extent of the Streptococcus pneumoniae infection in the patient from whom a sample was obtained. Further, detecting presence of the analyte in the vapor or gas sample (rather than in a liquid sample) improves the selectivity of the sensor against dissolved analyte that does not equilibrate with the vapor or gas phase sample.

### BRIEF DESCRIPTION OF THE DRAWINGS

FIG. 1 is a schematic and functional block-diagram of an exemplary setup and environment in which various aspects of the invention can be used;

FIG. 2 illustrates an exemplary electrochemical sensor used for detection of hydrogen peroxide according to various aspects of this invention;

FIG. 3 illustrates an exemplary experimental setup for testing the electrochemical sensor of FIG. 2 according to various aspects of this invention;

FIGS. 4A and 4B illustrate graphs of current versus voltage for detection of hydrogen peroxide according to the experimental setup of FIG. 3;

FIG. 5A illustrates a graph of current versus voltage for detection of hydrogen peroxide with ohmic drop reduced in the electrochemical sensor and FIG. 5B illustrates a logarithmic plot of the voltage output by the electrochemical sensor versus the concentration of hydrogen peroxide, according to various aspects of this invention;

FIG. 6A illustrates a graph of current versus voltage for detection of hydrogen peroxide with an acetate buffer added to the gelatinous coating on the electrochemical sensor and FIG. 6B illustrates a logarithmic plot of the voltage output by the electrochemical sensor versus the concentration of hydrogen peroxide according to various aspects of this invention; and

FIG. 7 is a flow chart of a method for detection of hydrogen peroxide according to various aspects of this invention.

While these examples are susceptible of embodiment in many different forms, there is shown in the drawings and will herein be described in detail various aspects of the invention, with the understanding that the present disclosure is to be considered as an exemplification and is not intended to limit the broad aspect of the embodiments illustrated in the drawings.

### DETAILED DESCRIPTION OF THE INVENTION

The present invention relates to electrochemical sensors and their use for the detection of an analyte present in a vapor or gas phase. As used herein, the term “vapor or gas phase” can be any such fluid sample, for example, a sample of exhaled breath that includes both vapor and gaseous components. The sample can be unmodified prior to analysis in accordance with the aspects of the present invention. As used herein, the “analyte” can be any electrochemically active analyte that partitions from the gas/vapor phase into a coating on the electrochemical sensor. Exemplary analytes include, without limitation, hydrogen peroxide, carbon dioxide, and ammonia.

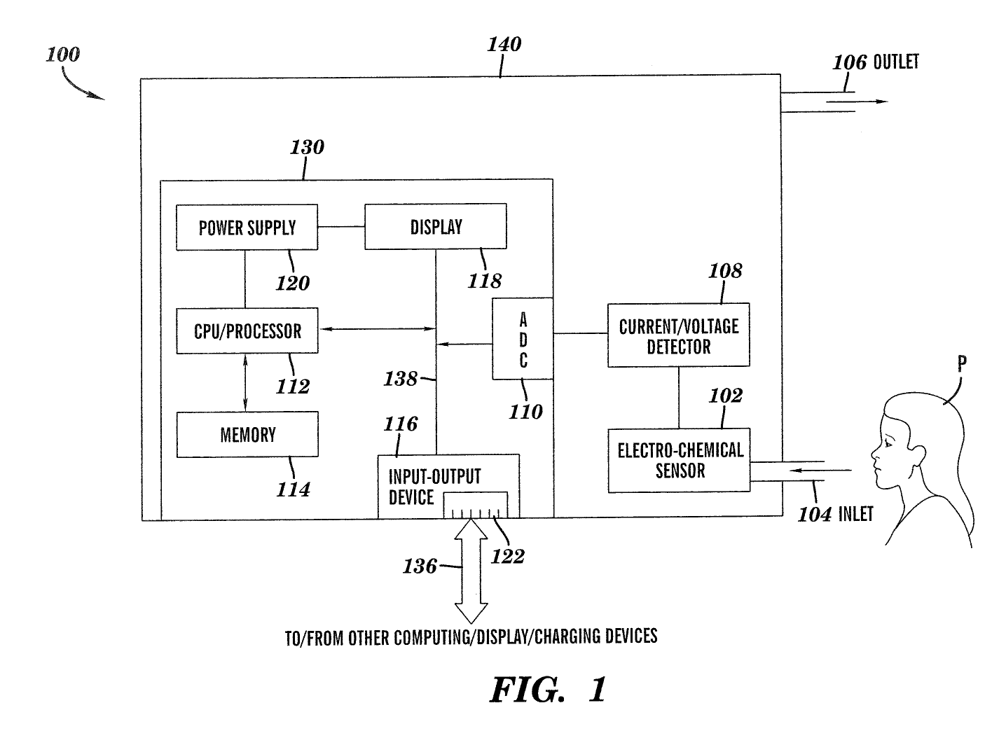

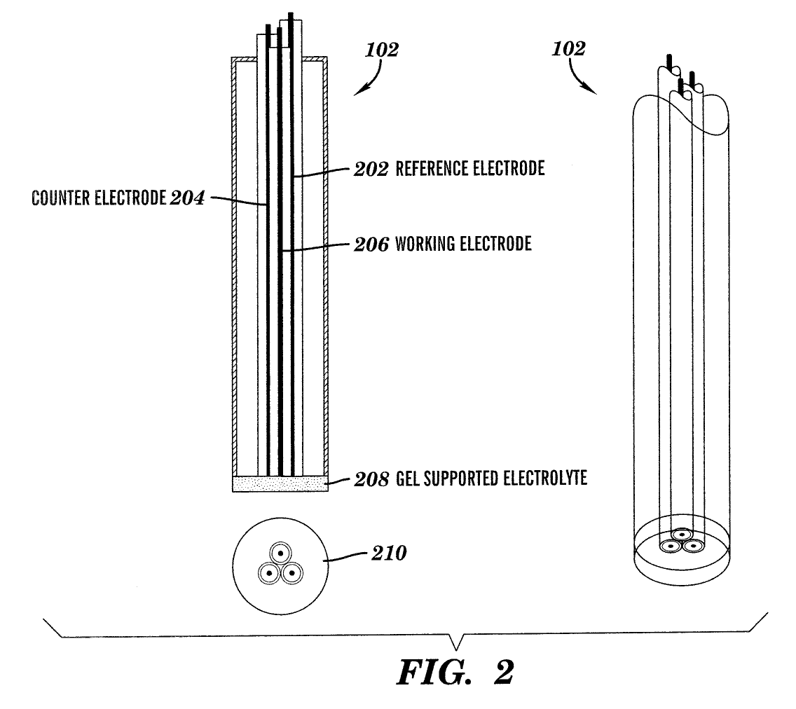

Referring to FIG. 1, an exemplary setup and environment 100 for detection of an analyte in a vapor or a gas phase is illustrated. In the setup and environment 100, a detector device 140 includes an inlet 104 through which a vapor or a gas sample is introduced into detector device 140 and an outlet 106 through which the inlet vapor or gas exits detector device 140. For example, through inlet 104, a patient P introduces expelled breath into detector device 140. To facilitate exposure to the vapor or gas sample, detector device 140 can include a mouthpiece that defines inlet 104 and/or outlet 106 through which patient P exhales, thereby causing the vapor or gas sample to pass over an electrochemical sensor 102 coupled to inlet 104. Alternatively, detector device 140 can be incorporated into a portion of an intubation tubing such that sensing of the exhaled vapor or gas can be achieved passively, i.e., without patient P actively participating in introducing the vapor or gas sample. According to yet another alternative aspect of the invention, detector device 140 may include only a single inlet/outlet but also includes expandable reservoir (e.g., a balloon) that receives and collects the expelled breath. After patient P fills the reservoir, the expelled breath is subsequently released through the single inlet/outlet while or after the received expelled breath is analyzed by detector device 140. It is to be noted that all example arrangements of inlet 104 and outlet 106 disclosed immediately above will allow hydrogen peroxide in the expelled breath to reach electrochemical sensor 102, and the various aspects of the invention are not limited by type of inlet 104 and/or outlet 106 used.

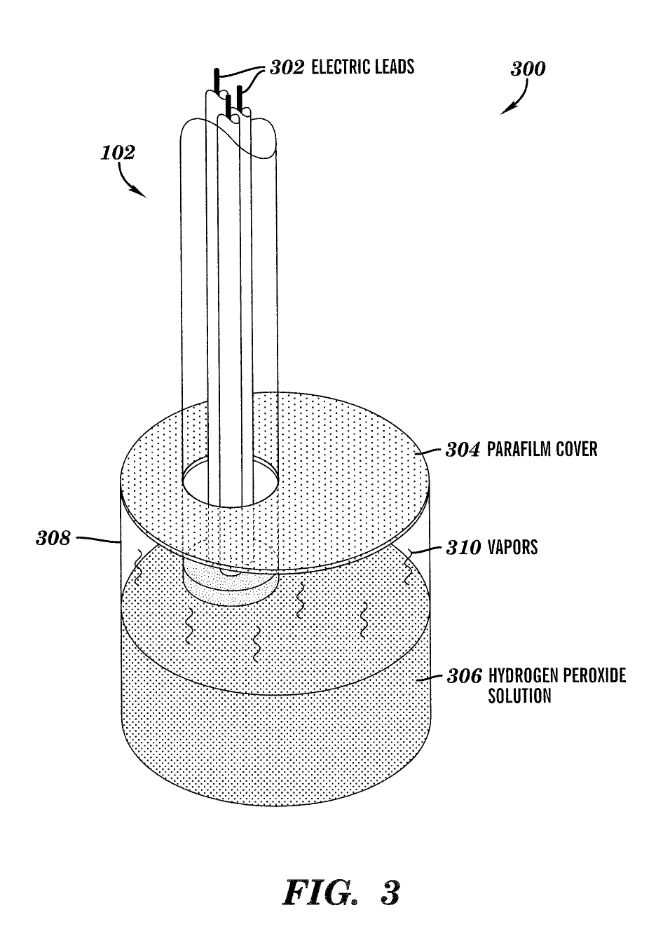

Electrochemical sensor 102 coupled to inlet 104 of detector device 140 receives the vapor or the gas sample in the exhaled breath. In response to the vapor or gas sample passing over electrochemical sensor 102, an output current is produced from a reduction-oxidation (redox) reaction at electrochemical sensor 102 as explained in more detail with respect to FIGS. 2 and 3 below. The construction and structure of electrochemical sensor 102 is described in more detail below in relation to FIG. 2. The amount of output current produced is in direct correlation to an amount of hydrogen peroxide present in the vapor or gas sample. The output current from electrochemical sensor 102 is coupled to a current/voltage detector 108 configured to filter unwanted frequencies from the output current, i.e., background current/voltage caused by ambient air. Optionally, depending upon specific applications, current/voltage detector 108 can convert the detected current output from electrochemical sensor 102 into a corresponding calibrated value, as will be apparent to those skilled in the art in view of this disclosure. Further, output from electrochemical sensor 102 may be directly fed to controller 130. The output of current/voltage detector 108 is a conditioned current substantially free from noise and other undesirable frequencies.

The conditioned current at the output of current/voltage detector is provided to an analog to digital converter (ADC) 110 inside controller 130. ADC 110 converts the analog output of current/voltage detector 108 to a corresponding digital value for processing by controller 130. The digital value of the detected current is provided to central processing unit (CPU)/processor 112 via an internal bus 138. By way of example only, ADC 110 can be an 8-bit ADC, although other types of ADCs may also be used as known to those skilled in the art.

CPU/processor 112 receives and processes the digital current from ADC 110. CPU/processor 112 can be a single board computer which includes one or more microprocessors or CPUs. Controller 130 may be conveniently implemented using one or more general purpose computer systems, microprocessors, digital signal processors, and micro-controllers, programmed according to the teachings described and illustrated herein. For example, CPU/processor 112 can be an Intel Core Duo® processor provided by Intel Corporation of Santa Clara, Calif. Alternatively, CPU/processor 112 may be a special purpose processor designed and fabricated to carry out various aspects of this invention. For example, CPU/processor 112 may be an application specific integrated circuit (ASIC) chip.

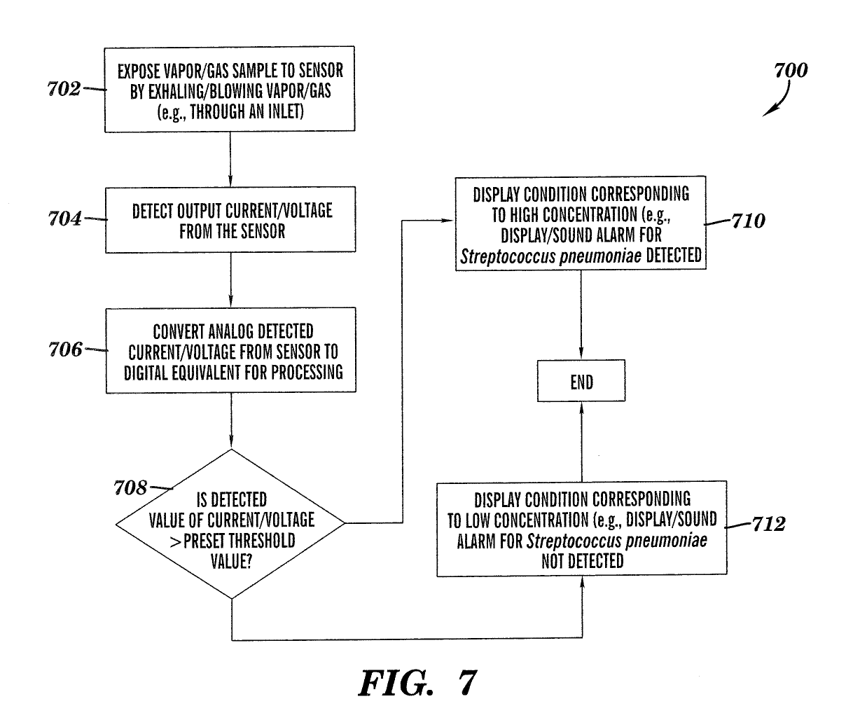

CPU/processor 112 is coupled to a memory 114 that stores various settings for detector device 140. For example, memory 114 stores a threshold value of the output current from electrochemical sensor 102. Memory 114 can be a random access memory (RAM) and/or read only memory (ROM), along with other conventional integrated circuits used on a single board computer as are well known to those of ordinary skill in the art. Alternatively or in addition, memory 114 may include a floppy disk, a hard disk, CD ROM, or other computer readable medium which is read from and/or written to by a magnetic, optical, or other reading and/or writing system that is coupled to one or more processors. Memory 114 can include instructions written in a computer programming language or software package for carrying out one or more aspects of the present invention as described and illustrated herein, although some or all of the programmed instructions could be stored and/or executed elsewhere. For example, instructions for executing steps outlined in FIG. 7 can be stored in a distributed storage environment where memory 114 is shared between one or more controllers similar to controller 130.

Controller 130 can include an input/output (I/O) device 116 (e.g., an I/O card) coupled to CPU/processor 112 and a display 118 via internal bus 138. I/O device 116 includes a bi-directional port 122 for communication to/from other computing and/or electronic devices via a link 136. By way of example only, I/O device 116 can be a keypad/keyboard that is capable of providing user inputs. Port 122 can also be used for charging detector device 140 when used in a mobile or wireless mode. By way of example only, port 122 can be a Universal Synchronous Bus (USB) port, although other types of communication and input/output ports may also be used, as known to those skilled in the art.

Internal bus 138 is designed to carry data, power and ground signals, as known to one skilled in the art. By way of example only, internal bus 138 can be a Peripheral Component Interconnect (PCI) bus, although other types of local buses (e.g., Small Computer System Interface or “SCSI”) may also be used, as known to those skilled in the art.

Display 118 can be a suitable display panel on which instructions and data are presented to a user in both textual and graphic format. In addition, display 118 can include a touch screen also coupled to I/O device 116 for accepting input from a user (e.g., a medical professional or patient P). Display 118 can display the concentration of the analyte in the vapor or gas sample based on the output current or voltage that is generated by electrochemical sensor 102. Further, display 118 may be substituted by or used in conjunction with an audio device (e.g., a speaker, a buzzer, or a beeper alarm) controlled by CPU/processor 112 to indicate various conditions resulting from patient P exhaling into inlet 104.

Controller 130 receives power from a power supply 120. Power supply 120 can be a battery or a direct pluggable outlet to a main power-line.

Alternatively, power supply 120 may be a switched mode power supply (SMPS) commonly used in computer systems, although other forms for powering controller 130 using power supply 120 may also be used, as known to those skilled in the art.

Using electrochemical sensor 102 of the present invention in combination with state of the art techniques for assessing Streptococcus pneumonia load, it is possible to generate empirical data that correlates detected conditioned current levels with the Streptococcus pneumonia counts. This empirical data can be used to form a model.

Based upon a model stored in memory 114 that correlates hydrogen peroxide concentration in the vapor/gas sample, i.e., detected signal levels from electrochemical sensor 102, to Streptococcus pneumonia infection, detector device 140 can also monitor and/or semi-quantitatively identify the severity of an infection in patient P. Output related to the severity of infection can also be displayed on display 118, for example. Alternatively, detector device 140 may sound an alarm or a beep to indicate that patient P has an infection or the severity of infection. That is, a higher measured hydrogen peroxide concentration, which correlates to a higher Streptococcus pneumonia load, may signal an alarm that differs in kind from a signal that corresponds to a lower measured hydrogen peroxide concentration.

In addition, various aspects of the present invention can also be used to monitor the sufficiency of a treatment of Streptococcus pneumoniae infection by assessing the concentration of hydrogen peroxide in exhaled breath both during and following a course of antibiotic treatment. Where an antibiotic has no effect even during a mid-course of a multi-day treatment regimen, it remains possible to monitor patient P's response and, if desired, switch therapies prior to completion of the particular course of therapy.

Various components of controller 130 (e.g., CPU/processor 112 along with memory 114) embody a computer readable medium having stored thereon instructions for determining an amount of an analyte (e.g., hydrogen peroxide) in a vapor or gas sample (e.g., patient P's exhaled breath). The instructions can include machine executable code which when executed by CPU/processor 112 causes CPU/processor 112 to perform steps of flowchart 700 described in FIG. 7 below and carry out the various methods disclosed herein. In addition, the computer readable medium can have instructions for making various decisions for operation of different aspects of the invention, including correlating the detected output current from electrochemical sensor with one or more values stored in memory 114 to determine whether patient P is infected with Streptococcus pneumoniae or not. Further, controller 130 can include other numbers and types of components, parts, devices, systems, and elements in other conventional components.

It is to be noted that although electrochemical sensor 102 is shown within detector device 140, various aspects of the present invention may equally be realized using a standalone electrochemical sensor 102 externally coupled to controller 130, as will be apparent to those skilled in the art in view of this disclosure. For example, one skilled in the art after reading this disclosure may modify the detector for trace level detection of analytes using artificial olfactometry as described in U.S. Pat. No. 6,244,096 to Lewis et al., which is hereby incorporated by reference in its entirety, to make a suitable detector that can include electrochemical sensor 102 of the present invention.

In addition, two or more computing systems or devices can be substituted for any one of the systems described above. Accordingly, principles and advantages of distributed processing, such as redundancy and replication, also can be implemented, as desired, to increase the robustness and performance of the devices and systems described above. The embodiments of the present invention may also be implemented on computer system or systems that extend across any suitable network using any suitable interface mechanisms and communications technologies, including, by way of example only, telecommunications in any suitable form (e.g., voice and modem), wireless communications media, wireless communications networks, cellular communications networks, G3 communications networks, Public Switched Telephone Networks (PSTNs), Packet Data Networks (PDNs), the Internet, intranets, and combinations thereof.

Referring to FIG. 2, an exemplary structure of electrochemical sensor 102 is illustrated. Element 210 illustrates a bottom plan view of electrochemical sensor 102. Electrochemical sensor 102 includes three micro-electrodes fabricated inside a borosilicate capillary structure with three compartments, although other numbers and types of electrodes, for example, two or four electrodes, may also be used. More specifically, according to one aspect of the invention, electrochemical sensor 102 includes a reference electrode 202, a counter electrode 204 and a working electrode 206, each of which have a tip at least one end surrounded with a coating 208 over which one or more molecules of an analyte pass. Alternatively, coating 208 may cover or surround more than the tip of reference electrode 202, counter electrode 204 and working electrode 206, for example, the whole of electrochemical sensor 102 could be embedded in the coating material.

Coating 208 is capable of selectively partitioning an electrochemically active analyte directly from the vapor or gas phase sample such that an oxidation/reduction current within coating 208 can be measured by two or more electrodes among reference electrode 202, counter electrode 204 and working electrode 206.

According to various aspects of the invention, a suitable structural component can be utilized in coating 208. The structural component can be polymeric or non-polymeric. Exemplary structural components include, without limitation, polyvinylchloride (PVC), silicone rubber, polyurethane, (meth)acrylate polymer, polypyrrole, polythiophene, polyoctylthiophene, polyanaline, polyvinyl pyrrolidone, agarose, hydrogel, (meth)acrylate gels, sol-gel materials, and combinations thereof.

According to other aspects of the invention, a suitable water immiscible organic solvent can be utilized in coating 208. The organic solvent is responsible for assisting in the partitioning of the analyte of interest from the vapor or gas sample into coating 208. Exemplary water immiscible organic solvents include, without limitation, 2-nitrophenyl octyl ether (o-NPOE), dioctyl sebacate (DOS), bis(2-ethylhexyl) sebacate, benzyl s-nitrophenyl ether, bis(1-butyipentyl) adipate, bis(2-ethylhexyl)adipate, bis(2-ethylhexyl)phthalate, 1-chloronaphthalene, chloroparaffin, 1-decanol, dibutyl phthalate, dibutyl sebacate, dibutyl-dilaurate, dodecyl 2-nitrophenyl ether, and combinations thereof.

According to another aspect, a suitable charge transfer agent can be utilized in coating 208. Exemplary charge transfer components include, without limitation, tetradecylammonium tetrakis(pentofluorophenyl)borate (TDATPFPB), tetrahexylammonium perchlorate, and combinations thereof.

According to another aspect, a suitable membrane resistance controlling agent can be utilized in coating 208, when desired. Exemplary membrane resistance controlling agents include, without limitation, lipophilic electrolytes, tetradodecyl ammonium-tetrakis(4-chlorophenyl) borate (ETH500), bis(triphenylphoranylidene) ammonium tetrakis[3,5-bis(trifluoromethyl)phenyl] borate (BTPPATFPB), and combinations thereof.

According to another aspect, a suitable biocompatibility enhancing component can be utilized in coating 208, when desired. Exemplary biocompatibility enhancing components include, without limitation, nitric-oxide releasing sol-gel materials, N-(6-aminohexyl)aminopropyltrimethoxysilane, balanced isobutyltrimethoxysilane diazeniumdiolate, and combinations thereof.

According to another aspect, coating 208 is formed from a composition including about 15 to about 67 weight percent PVC, about 33 to about 85 wt percent o-NPOE, and about 0.001 to about 15 weight percent TDATPFPB.

According to another aspect, coating 208 can contain a structural component, a water and glycerol mixture, and salt. The mixture of glycerol and water is intended to reduce the evaporative loss of water. Coating 208 may optionally contain one or more further additives including, without limitation, a charge transfer component, a membrane resistance controlling component, and a biocompatibility enhancing component as described above. Any of the above-identified structural components can be utilized, preferably polyvinylchloride (PVC), (meth)acrylate gels, agarose, hydrogel, sol-gel materials, and combinations thereof. By way of example only, the glycerol-to-water ratio is at least about 1:3, more preferably about 1:4 up to about 1:20, and most preferably about 1:5 up to about 1:10, although other ratio values may also be used, depending upon specific applications, as known to those skilled in the art.

Coating 208 can be of a suitable dimension that affords effective partitioning while allowing for sufficient oxidation/reduction current within coating 208. For example, and not by limitation, coating 208 is less than about 200 μm thick, more preferably less than about 100 μm thick. According to one embodiment, coating 208 has a sub-micron thickness. According to another embodiment, coating 208 is between about 1 to about 25 μm thick. According to various aspects of the invention, the thickness of coating 208 can be optimized (e.g., by maintaining a constant salt concentration) for replication and for keeping the peak output current constant.

Reference electrode 202, counter electrode 204 and working electrode 206 can be formed out of a suitable conductive material including, without limitation, carbon, gold, platinum, palladium, ruthenium, rhodium or combinations thereof. Although only three microelectrodes—reference electrode 202, counter electrode 204 and working electrode 206 are described with respect to FIG. 2, according to certain embodiments four electrodes can be present. Further, various aspects of the invention are not limited by specific arrangement and structure of reference electrode 202, counter electrode 204 and working electrode 206 shown in FIG. 2, and one skilled in the art after reading this disclosure may devise other arrangements and structures. Exemplary electrode functions include, working electrode, auxiliary or counter electrode, and reference electrode. The particular function and number of electrodes will depend upon the type of electrochemical sensor 102 that is employed, and aspects of the present invention are not limited by specific formation(s) of electrochemical sensor 102.

Exemplary electrochemical sensor 102 types include, without imitation, voltammetric sensors, potentiometric sensors, conductometric sensors, and coulometric sensors. A voltammetric sensor can include, without limitation, one or more working electrodes (e.g., working electrode 206) in combination with reference electrode 202, or one or more working electrodes (e.g., working electrode 206) in combination with reference electrode 202 and counter electrode 204, as shown in FIG. 2. In voltammetry, the potential applied to working electrode 206 is varied over time to measure the current through coating 208.

Alternatively, a conductometric sensor can include two or four electrodes, which measure the impedance of the coating; a potentiometric cell can include two electrodes, in which the potential of the indicator electrode is measured at zero current; and a coulometric sensor can include two or more electrodes. The design and principles surrounding these types of electrochemical sensors are described, for example, in Toth et al., “Electrochemical Detection in liquid Flow Analytical Techniques: Characterization and Classification,” Pure Appl. Chem. 76(6):1119-1138 (2004), which is hereby incorporated by reference in its entirety.

According to a further aspect of the invention, the electrochemical sensor can be incorporated into a microfluidic sensor that includes a microfluidic channel and coated electrode(s) positioned with coating 208 in communication with the microfluidic channel through which the vapor or gas sample passes during the detection procedure. Such a microfluidic sensor can be used to assess the presence or quantity of the analyte of interest in the sample.

### EXAMPLES

The Examples set forth below are for illustrative purposes only and are not intended to limit, in any way, the scope of the present invention.

### Example 1

### Construction of Sensors

A prototype sensor according to FIG. 2 was fabricated using working electrode 206 formed of a 25 μm diameter platinum wire, counter electrode 204 formed of a cluster of 8 μm diameter carbon fiber, and reference electrode 202 formed of a 75 μm silver wire. According to an exemplary fabrication technique for electrochemical sensor 102, a capillary holding working electrode 206, counter electrode 204, and reference electrode 202 was sealed at one end over a Bunsen burner. Electrode wires were then placed in separate compartments, and the capillary placed in a heating coil under vacuum to collapse the glass and seal the electrode wires. Each of the working electrode 206, counter electrode 204, and reference electrode 202 was polished using fine sandpaper and a polishing pad with silicon paste to expose a surface of the three electrodes. Connections to working electrode 206, counter electrode 204, and reference electrode 202 were made using electrical wire held by silver epoxy and the electrodes were tested for connectivity in a 0.1 M phosphate buffer solution.

Coating 208 was optimized to maximize salt content, spreading, and solidity, and to minimize moisture loss. Agar gel (provided, for example, by Fischer Scientific of Pittsburgh, Pa., which has a low gelation temperature) in water (acting as a solvent) was used to form coating 208. To increase drying time of the gel for better charge flow, a mixture of glycerol and water was used in different ratios (2:1, 1:1, 1:2, 1:3, and 1:5). The glycerol:water mixtures were saturated with sodium nitrate to determine their loading capacity prior to adding Agar gel in powder form. As demonstrated by the results below, of these mixtures tested, a ratio of 1:5 was preferred, because the higher water content supports maximum salt loading, the mixture is substantially fluid, and is easy to spread on the electrode surface. A higher concentration of salt used for coating 208 facilitates a lower resistance to the redox current and consequently provides a higher observed current output by electrochemical sensor 102. Unfortunately, a high concentration of salt can also interfere with gelation of coating 208. By way of example only, an unsaturated solution of 3M sodium nitrate in the 1:5 glycerol to water solvent can be used to make a substantially effective gel for purposes of various aspects of this invention. During gelation, some water may be lost, creating a higher salt concentration as compared to a nominal value of salt concentration in the gel before coating 208, on which the gel resides, can be tested.

Upon the oxidation of H₂O₂ to O₂ on working electrode 206 (made of platinum, for example), H⁺ ions are generated. To prevent the continuous acidification of coating 208 in repeated and/or continuous measurements, coating 208 was buffered. Therefore, according to one aspect, a supporting electrolyte can be introduced into the gel in the form of a 1M phosphate buffer or an acetate buffer. An acetate buffer can be introduced in the gel (in addition to the 3M NaNO₃) by adding 0.01M acetic acid and 0.005M sodium hydroxide.

In an alternative construction, a PVC membrane can also be used to determine the effect of a more hydrophobic membrane on hydrogen peroxide detection. According to one aspect of the invention, the PVC membrane was cast over the surface of working electrode 206 from a diluted tetrahydrofuran (THF) solution. A few microliters of THF solution can be dispensed over the surface of the planar electrochemical cell using, for example, a microsyringe. By way of example only, the composition of the THF solution used was 60 mg PVC, 120 mg (2-nothrophenyl octylether (o-NPOE) as plasticizer, in 2.0 mL THF. The PVC membrane formed over the surface of working electrode 206, counter electrode 204, and/or reference electrode 202 after the THF evaporated.

### Example 2

### Experimental Detection Chamber

Referring to FIG. 3, an experimental setup 300 to determine whether the agar or PVC coating provides a medium where electrochemical experiments can be performed is illustrated. The experimental setup 300 included electrochemical sensor 102 inserted into a beaker 308 filled at least partially with a hydrogen peroxide solution 306. Electrochemical sensor 102 passes through a parafilm cover 304 covering an open top of 20 mL beaker 308 such that coating 208 of electrochemical sensor 102 is exposed to vapors 310 emanating from hydrogen peroxide solution 306.

The concentration of the hydrogen peroxide solution 306 was varied between the cyclic voltammetric scans by diluting with distilled, deionized water and stirring with a magnetic stir bar.

### Example 3

### Detection of Hydrogen Peroxide

A cyclic voltammetric scan to a negative potential was performed to observe the signal from the reduction of atmospheric oxygen in vapors 310 over coating 208. Once charge transport was detected from electrochemical sensor 102 (e.g., by current/voltage detector 108 coupled to electrical leads 302), cyclic voltammetric scans were performed with potentials ranging from 0 to 1.4 V and 0 to 1.2 V with a scan rate of 0.02 V/s and a sample interval of 0.001 V.

Measurements can be made inside a Faraday cage to insulate experimental setup 300 from external and/or undesirable static charges (e.g., using a potentiostat provided by CH Instruments of Austin, Tex.). In addition to cyclic voltammetry other voltammetric techniques can also be used, e.g., chronoamperometry, pulse voltammetry, differential pulse voltammetry, square wave voltammetry. Although in these techniques the calculation of the Faradic current is different, it does not affect the various aspects of this invention. In addition, the background scan can be taken in air and subtracted from the collected data to account for Faraday currents in working electrode 206, counter electrode 204, and reference electrode 202, without affecting the various aspects of this invention.

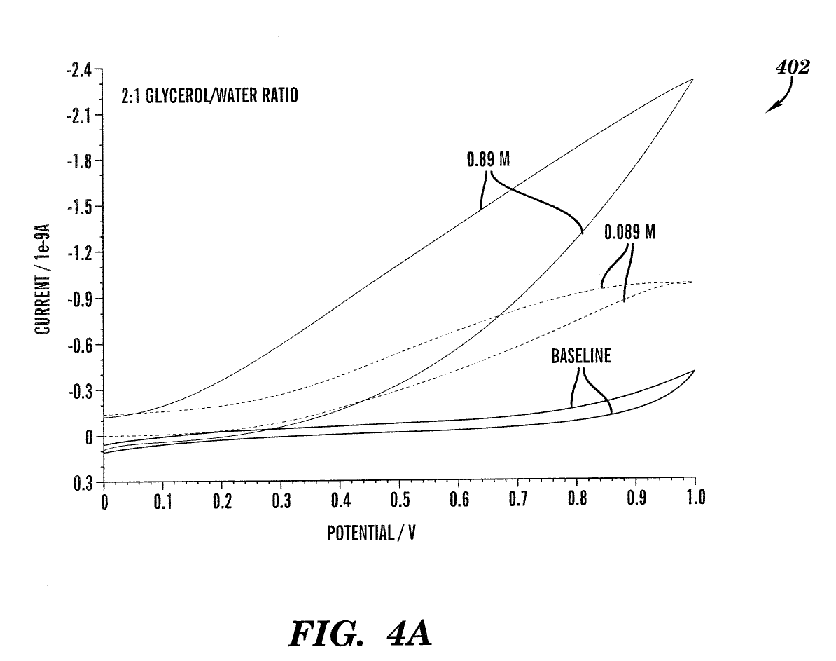

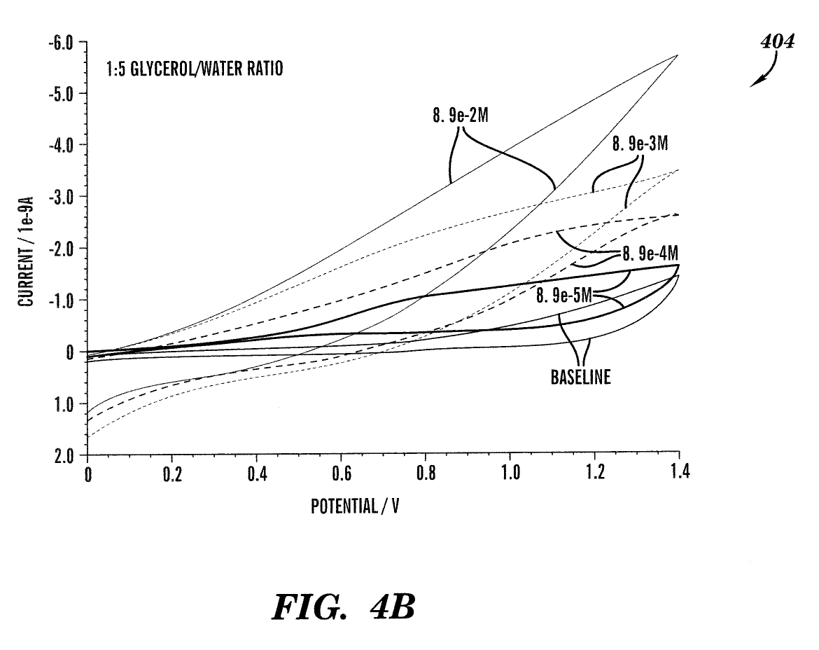

Thereafter, voltammetric scans were performed in the gas phase, above solutions having varying concentrations of hydrogen peroxide. Referring to FIGS. 4A, 4B, 5A, 5B, 6A, and 6B, results of different experiments conducted using experimental setup 300 are illustrated. It is to be noted that one skilled in the art can contemplate other conditions for testing electrochemical sensor 102 apart from the condition described above. A voltammogram for experimental setup 300 results in a background current output from electrical leads 302 when a potential was applied to working electrode 206, both with the agar and the PVC membrane forming coating 208. Exposure to hydrogen peroxide in vapors 310 surrounding coating 208 causes an increase in the current measured by electrochemical sensor 102.

The results demonstrate that the current observed in the agar gel is higher than the current in the PVC membrane due to the hydrophobicity of the PVC. As a result, agar was used primarily as the medium for a supporting electrolyte for the remainder of the experiments. The results presented herein demonstrate that as the concentration of hydrogen peroxide in vapors 310 around and below electrochemical sensor 102 increased, the measured current from electrical leads 302 also increased.

Referring to FIG. 4A results from experimental setup 300 with a 2:1 glycerol/water ratio with 1M NaNO₃ for coating 208, over 0.89 M and 0.089 M stock hydrogen peroxide solution 306 corrected for baseline are shown. In FIG. 4B, results from experimental setup 300 with an optimized agar gel including a 1:5 glycerol/water ratio with 3M NaNO₃ over 8.9e-2M, 8.9e-3M, 8.9e-4M, and 8.9e-5M, in a top to bottom arrangement, stock hydrogen peroxide solution 306 corrected for baseline (blue) are shown. As can be seen from the plots in FIGS. 4A and 4B, current output by electrical leads 302 of electrochemical sensor 102 increases as concentration of hydrogen peroxide in hydrogen peroxide solution 306 increases.

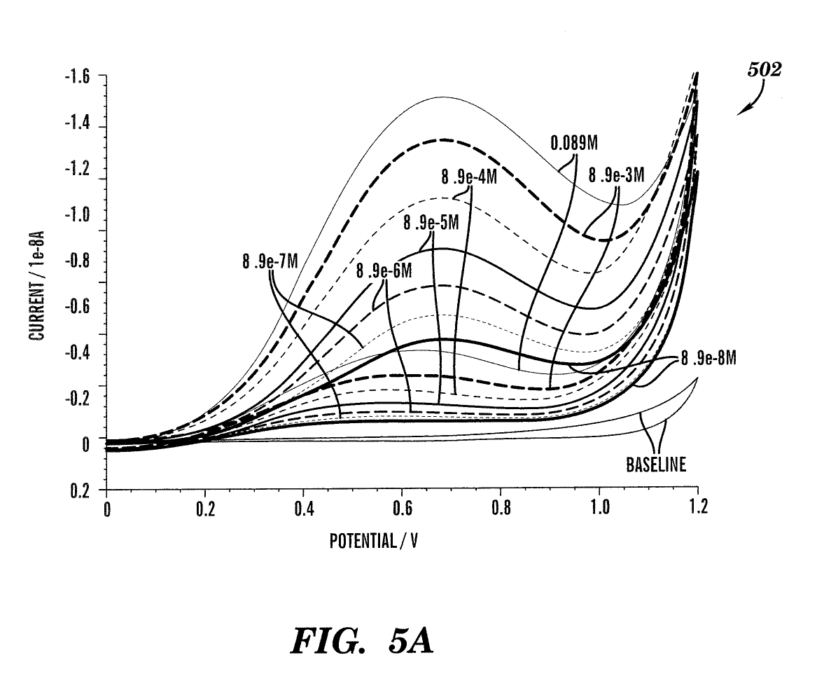

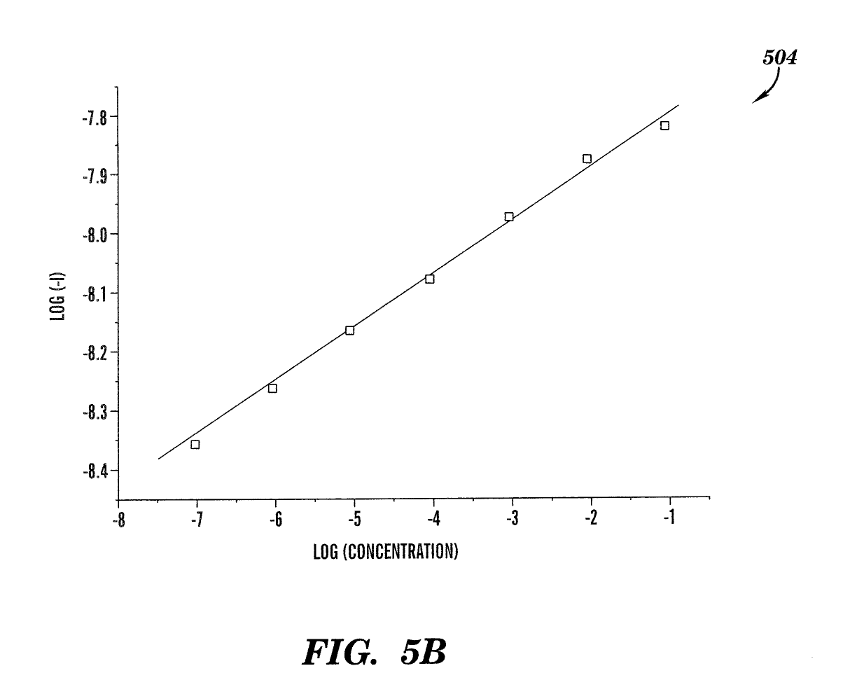

Referring more specifically to FIGS. 5A and 5B, results using experimental setup 300 with a different value for concentration of hydrogen peroxide solution 306 are shown. FIG. 5A illustrates a scenario with coating 208 comprising 3M NaNO₃ over 0.089 M, 8.9e-3 M, 8.9e-4 M, 8.9e-5 M, 8.9e-5 M, 8.9e-7 M, and 8.9e-8 M concentrations of hydrogen peroxide solution 306. As seen in FIG. 5A, current output from electrical leads 302 of electrochemical sensor 102 varies as potential across reference electrode 202 and working electrode 206 is varied.

FIG. 5B illustrates a graph showing direct correlation between the current output by electrical leads 302 and the concentration of hydrogen peroxide in solution 306 when a logarithm of the current was plotted as a function of a logarithm of the concentration of hydrogen peroxide solution 306 at a fixed voltage potential of 0.7 V, although other values of voltage may also be used as known to those skilled in the art.

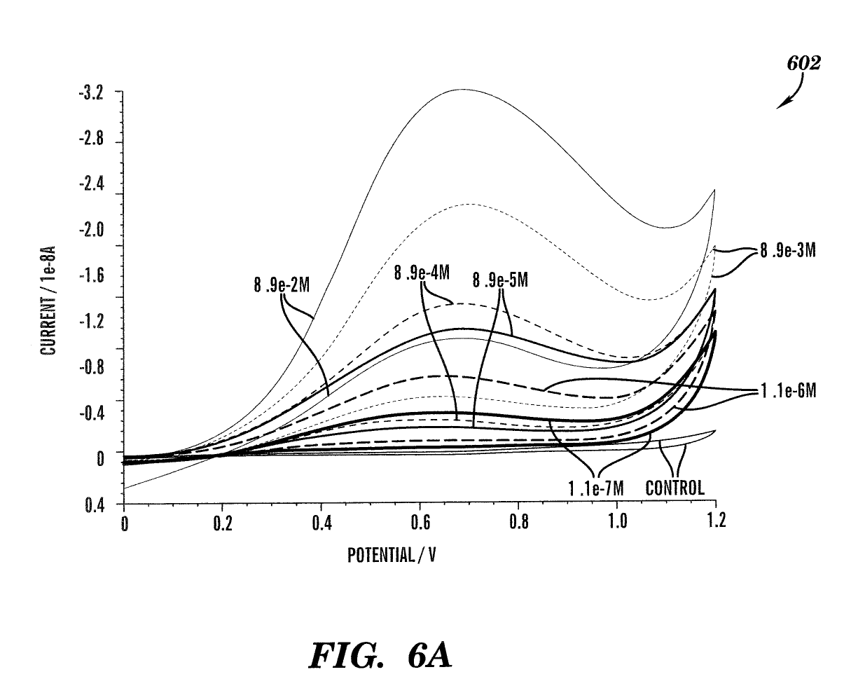

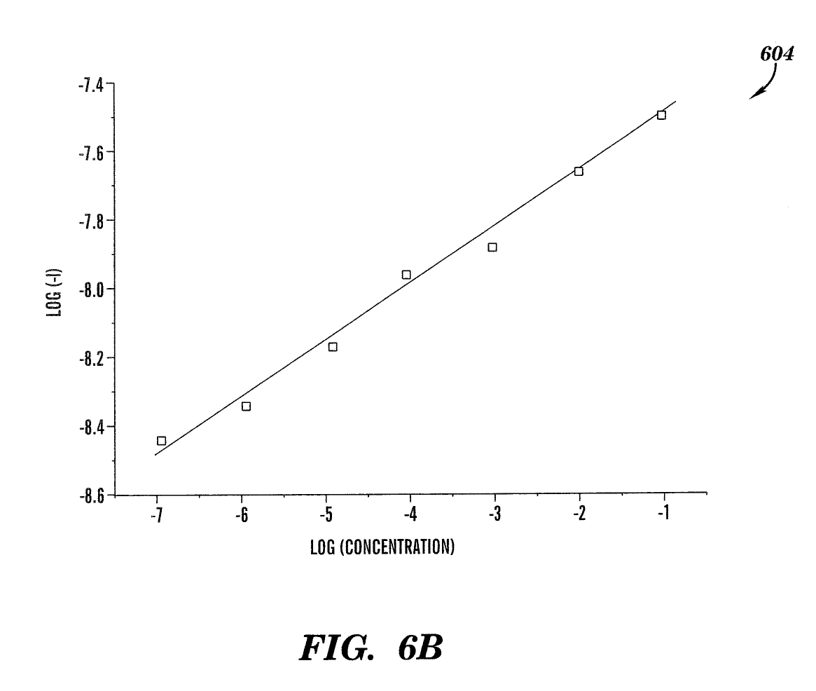

Referring to FIGS. 6A and 6B, the effects of acetate buffer on coating function is illustrated (agar coating contained 3M NaNO₃ and acetate buffer). Because the electron transfer half reaction of hydrogen peroxide with platinum of working electrode 206 produces two charged hydroxide ions, the pH inside parafilm cover 304 can be variable during the electrochemical scan. In FIG. 6A, the resulting current following exposure to hydrogen peroxide concentrations of 0.089M, 8.9e-3 M, 8.9e-4 M, 8.9e-5M, 1.1e-6 M, and 1.1e-7 M are illustrated. As shown in FIG. 6A, current output from electrical leads 302 of electrochemical sensor 102 varies as potential across reference electrode 202 and working electrode 206 is varied. FIG. 6B illustrates a graph where a logarithm of current output by electrical leads 302 versus a logarithm of concentration of hydrogen peroxide solution 306 at a fixed voltage of 0.7 V. Similar to the graph of FIG. 5B, the dependence of output current in direct proportion to the concentration of hydrogen peroxide in hydrogen peroxide solution 306 can be observed.

Based on the current profile measured in air, it is apparent from FIGS. 4A-6B that the agar and the PVC are viable substitutes for a conventional supporting electrolyte medium for electrochemistry. When hydrogen peroxide was present in vapors 310, a significant current was detected in the potential range for hydrogen peroxide, and the analyte in the vapor or gas phase could partition into and move through coating 208 to the surface of working electrode 206, reference electrode 202 and counter electrode 204, allowing for electron transfer to take place and cause charge flow.

Because the partitioned analyte concentration in coating 208 is in equilibrium with the vapor (or gas) phase, i.e., the analyte concentration in coating 208 is proportional to the concentration in the vapor or gas phase, a reliable assessment can be made concerning the analyte concentration in the vapor or gas phase. In addition, by correlating the vapor or gas phase analyte concentration to the severity of Streptococcus pneumoniae, it is possible to assess the extent of the Streptococcus pneumoniae infection in patient P from whom a sample was obtained.

Referring to FIG. 7, a flowchart 700 illustrates exemplary steps for a method for electrochemical detection of an analyte in a vapor or gas sample, as described with respect to FIGS. 1-6B above, according to one aspect of the invention. According to another aspect of the invention, steps 702-710 can be used in a method for detecting a Streptococcus pneumoniae infection in patient P, as described with respect to FIGS. 1-6B above. Further, steps 702-710 discuss the operation of electrochemical sensor 102 used according to various aspects of the present invention.

In step 702, electrochemical sensor 102 is exposed to a vapor or a gas sample (e.g., vapors 310). The exposing can be performed, for example, by patient P blowing or exhaling at a mouthpiece attached to inlet 104. Alternatively, electrochemical sensor 102 may be exposed to an analyte (e.g., hydrogen peroxide) in other setups, for example, experimental setup 300 in FIG. 3.

In step 704, based upon exposure to the analyte in the vapor or gas sample, electrochemical sensor 102 outputs a current (or equivalently, a voltage) from electrical leads 302 as a result of a redox reaction on coating 208. By way of example only, the output current can be detected by current/voltage detector 108 of detector device 140. Alternatively, the output current may be directly fed to controller 130 for further processing or detected by other current/voltage detection schemes, which are well known to those skilled in the art.

In step 706, controller 130 compares the value of the output current or voltage detected with a preset threshold value stored in memory 114 of controller 130. The preset threshold value may correspond to a desired level of analyte concentration in the vapor or gas sample and the amount of output current detected can be correlated to a corresponding amount of the analyte concentration by controller 130. Based upon the correlating and comparison with the preset threshold, controller 130 determines if the value of analyte concentration is higher than the preset threshold value stored in memory 114.

If the value of detected current/voltage is higher than the preset threshold, the flow proceeds to step 708 in which controller 130 displays a condition corresponding to a high concentration of the analyte. For example, controller 130 can show a presence of Streptococcus pneumoniea in the upper respiratory system of patient P if high amounts of hydrogen peroxide are present in the vapor or gas sample received by detector device 140. Alternatively, controller 130 may display other conditions based upon the comparison, for example, presence of undesirable additives in a hydrogen peroxide sample.

However, if the value of detected current/voltage is lower than the preset threshold, the flow proceeds to step 708 in which controller 130 displays a condition corresponding to a low concentration of the analyte. For example, such a condition can be used as a diagnostic tool to test whether or not patient P is free from Streptococcus pneumoniea infection and to infer whether or not, even with a low concentration of hydrogen peroxide, patient P is developing a Streptococcus pneumoniea infection by monitoring patient P on a regular or a random basis.

It is to be noted that although display 130 is being used to show conditions corresponding to high or low concentrations of the analyte, other forms of indication such as a beep, a buzzer, or a flashing light may also be used, as known to one skilled in the art.

Various aspects of the present invention provide advantages over conventional detection of analytes in vapor phase, and more specifically over conventional methods and devices for detecting undesirable populations of Streptococcus pneumoniea. For example, using experimental setup 300 it is possible to use electrochemical techniques to detect the presence of hydrogen peroxide in air by condensing the electrochemical cell to one unit and applying a membrane layer over the tip of the cell as the supporting electrolyte. In addition, as shown in FIGS. 4A-6B, the current measured with this technique has a clear linear dependence on the concentration of the ambient hydrogen peroxide in the air surrounding electrochemical sensor 102. Using various aspects of the present invention, it is possible to detect the hydrogen peroxide concentration in the breath of patient P produced by Streptococcus pneumoniea in the upper respiratory system. Detection of hydrogen peroxide in this way provides an efficient noninvasive diagnostic tool to monitor S. pneumoniea levels in individuals at high risk for infection, for example, young children, elderly persons, and the immunocompromised.

The results of experimental setup 300 conclusively show that coating the surface of working electrode 206, reference electrode 202 and counter electrode 204 with a hydro-gel or polymeric membrane allows electrochemistry to be used to detect the presence of hydrogen peroxide in the gaseous or vapor phase. In addition, a linear correlation between the concentration of hydrogen peroxide in air surrounding electrochemical sensor 102 and the observed current shows that electrochemical sensor 102 is sensitive to the concentration as well as the presence of hydrogen peroxide. This indicates that electrochemical sensor 102 can be used to quantify gaseous or vapor phase hydrogen peroxide in a sample, and particularly for purposes of detecting the presence of hydrogen peroxide in the exhaled breath of patients having a Streptococcus pneumoniea infection. It is also possible to use the concentration of hydrogen peroxide in breath as an indirect measure of the severity of infection—i.e., Streptococcus pneumoniea population size, for example, in an Streptococcus pneumoniea culture, or otherwise.

In addition, variations in the materials for working electrode 206, coating 208 and dimensions thereof can be assessed as part of the optimization process. Due to the delicate properties of agar gel used in exemplary coating 208, additional materials can also be used, such as sol gels, polymeric substances like polymethylmethacrylate, and other hydrogel materials. While the PVC membrane is functional, modifications to the PVC membrane with variations of electrode materials to can be made to optimize sensitivity to analyte concentrations in vapor or gas phase without undue experimentation by those skilled in the art.

All of the features and aspects of the present invention described herein (including any accompanying claims, abstract and drawings), and/or all of the steps of any method or process so disclosed, may be combined with any of the above aspects in any combination, except combinations where at least some of such features and/or steps are mutually exclusive.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. Additionally, the recited order of processing elements or sequences, or the use of numbers, letters, or other designations therefore, is not intended to limit the claimed processes to any order except as may be specified in the claims. These alterations, improvements, and modifications are intended to be suggested hereby, and are within the spirit and scope of the invention. Accordingly, the invention is limited only by the following claims and equivalents thereto.

What is claimed:

1. A method for electrochemical detection of an analyte in a vapor or gas sample, the method comprising:
exposing a vapor or gas sample to an electrochemical sensor comprising one or more electrodes and a coating that surrounds the one or more electrodes, which coating partitions the analyte directly from the vapor or gas sample; and
detecting an oxidation-reduction current during said exposing, wherein the detected current relates to a concentration of the analyte in the vapor or gas sample.

2. The method according to claim 1, wherein the analyte is H₂O₂.

3. The method according to claim 1, wherein the electrochemical sensor is a voltammetric sensor, a potentiometric sensor, a conductometric sensor, or a coulometric sensor.

4. The method according to claim 1, wherein the coating comprises a structural component, a water immiscible organic solvent, and a charge transfer component.

5. The method according to claim 4, wherein the structural component comprises a polymer selected from the group of polyvinylchloride (PVC), silicone rubber, polyurethane, (meth)acrylate polymer, agarose, hydrogel, or combinations thereof.

6. The method according to claim 4, wherein the water immiscible organic solvent comprises 2-nitrophenyl octyl ether (o-NPOE), dioctyl sebacate (DOS), bis(2-ethylhexyl)sebacate, benzyl s-nitrophenyl ether, bis(1-butilpentyl)adipate, bis(2-ethylhexyl)adipate, bis(2-ethylhexyl)phthalate, 1-chloronaphthalene, chloroparaffin, 1-decanol, dibutyl phthalate, dibutyl sebacate, dibutyl-dilaurate, dodecyl 2-nitrophenyl ether, or combinations thereof.

7. The method according to claim 4, wherein the charge transfer component is tetradecylammonium tetrakis(pentofluorophenyl)borate (TDATPFPB), tetrahexylammonium perchlorate, or combinations thereof.

8. The method according to claim 4, wherein the coating comprises about 15 to about 67 weight percent PVC, about 33 to about 85 weight percent o-NPOE, and about 0.001 to about 15 wt percent TDATPFPB.

9. The method according to claim 1, wherein the coating comprises a structural component, a mixture of glycerol and water, and a salt.

10. The method according to claim 9, wherein the structural component comprises a polymer selected from the group of polyvinylchloride (PVC), silicone rubber, polyurethane, (meth)acrylate polymer, polypyrrole, polythiophene, polyoctylthiophene, polyanaline, polyvinyl pyrrolidone, agarose, hydrogel, (meth)acrylate gel, sol-gel materials, or combinations thereof.

11. The method according to claim 9, wherein the mixture of glycerol and water comprises a glycerol-to-water ratio of at least about 1:3.

12. The method according to claim 9, wherein the salt is a nitrate, an acetate, a phosphate, or a combination thereof.

13. The method according to claim 1, wherein the coating comprises agar, mixture of glycerol and water, and a nitrate salt.

14. A method for detecting a Streptococcus pneumoniae infection in a patient, the method comprising:
exposing a patient breath sample to an electrochemical sensor comprising one or more electrodes and a coating that surrounds the one or more electrodes, which coating partitions hydrogen peroxide directly from the patient breath sample; and
detecting an oxidation-reduction current during said exposing, wherein the detected current relates to a concentration of hydrogen peroxide in the patient breath sample, the concentration of hydrogen peroxide indicating the extent of the Streptococcus pneumoniae infection.

15. The method according to claim 14, wherein the electrochemical sensor is a voltammetric sensor, a potentiometric sensor, a conductometric sensor, or a coulometric sensor.

16. The method according to claim 14, wherein the coating comprises a structural component, a water immiscible organic solvent, and a charge transfer component.

17. The method according to claim 16, wherein the structural component comprises a polymer selected from the group of polyvinylchloride (PVC), silicone rubber, polyurethane, (meth)acrylate polymer, agarose, hydrogel, or combinations thereof.

18. The method according to claim 16, wherein the water immiscible organic solvent comprises 2-nitrophenyl octyl ether (o-NPOE), dioctyl sebacate (DOS), bis(2-ethylhexyl)sebacate, benzyl s-nitrophenyl ether, bis(1-butilpentyl)adipate, bis(2-ethylhexyl)adipate, bis(2-ethylhexyl)phthalate, 1-chloronaphthalene, chloroparaffin, 1-decanol, dibutyl phthalate, dibutyl sebacate, dibutyl-dilaurate, dodecyl 2-nitrophenyl ether, or combinations thereof.

19. The method according to claim 16, wherein the charge transfer component is tetradecylammonium tetrakis(pentofluorophenyl)borate (TDATPFPB), tetrahexylammonium perchlorate, or combinations thereof.

20. The method according to claim 14, wherein the coating comprises a structural component, a mixture of glycerol and water, and a salt.

21. The method according to claim 20, wherein the structural component comprises a polymer selected from the group of polyvinylchloride (PVC), silicone rubber, polyurethane, (meth)acrylate polymer, polypyrrole, polythiophene, polyoctylthiophene, polyanaline, polyvinyl pyrrolidone, agarose, hydrogel, (meth)acrylate gel, sol-gel materials, or combinations thereof.

22. The method according to claim 20, wherein the mixture of glycerol and water comprises a glycerol-to-water ratio of at least about 1:3.

23. The method according to claim 20, wherein the salt is a nitrate, an acetate, a phosphate, or a combination thereof.

24. The method according to claim 14, wherein said detecting is repeated periodically.

25. The method according to claim 14, wherein said detecting is repeated periodically following administration of an antibiotic treatment to the patient.

26. An electrochemical sensor comprising two or more electrodes, and a coating that surrounds the two or more electrodes configured to selectively partition an electrochemically active analyte from a vapor or a gas phase such that an oxidation/reduction current within the coating can be measured.

27. The electrochemical sensor according to claim 26, wherein the analyte is hydrogen peroxide.

28. The electrochemical sensor according to claim 26, wherein the electrochemical sensor is a voltammetric sensor, a potentiometric sensor, a conductometric sensor, or a coulometric sensor.

29. The electrochemical sensor according to claim 26, wherein the coating comprises a structural component, a mixture of glycerol and water, and a salt.

30. The electrochemical sensor according to claim 29, wherein the structural component comprises a polymer selected from the group of polyvinylchloride (PVC), silicone rubber, polyurethane, (meth)acrylate polymer, polypyrrole, polythiophene, polyoctylthiophene, polyanaline, polyvinyl pyrrolidone, agarose, hydrogel, sol-gel materials, or combinations thereof.

31. The electrochemical sensor according to claim 29, wherein the mixture of glycerol and water comprises a glycerol-to-water ratio of at least about 1:3.

32. The electrochemical sensor according to claim 29, wherein the salt is nitrate, an acetate, a phosphate, or a combination thereof.

33. The electrochemical sensor according to claim 26, wherein the coating comprises agar, mixture of glycerol and water, and a nitrate salt.
